(19) United States
(12) Patent Application Publication
(10) Pub. No.: US 2016/0263026 A1
(43) Pub. Date: Sep. 15, 2016

(54) ORAL THIN FILM FORMULATION FOR REDUCTION OF VISION LOSS FROM MACULAR DEGENERATION

(71) Applicant: MACULAR HEALTH, LLC, Gardendale, AL (US)

(72) Inventors: Jeffery MCANNALLY, Morris, AL (US); John O. MASON, III, Birmingham, AL (US)

(21) Appl. No.: 15/035,947

(22) PCT Filed: Nov. 12, 2014
(86) PCT No.: PCT/US14/65263

§ 371 Date: May 11, 2016

Related U.S. Application Data
(60) Provisional application No. 61/903,655, filed on Nov. 13, 2013.

(51) Int. Cl. A61K 9/00; A61K 31/047; A61K 33/30

(52) U.S. Cl. CPC A61K 9/0056; A61K 33/30; A61K 31/047

(57) ABSTRACT

An oral thin film for inhibiting the progression of macular degeneration, having as active ingredients a zinc compound, lutein, or zeaxanthin, or a combination thereof, contained in an ingestible oral thin film. The oral thin film is placed in the mouth of a user and allowed to dissolve therein. The zinc compound, lutein, or zeaxanthin, or a combination thereof, is ingested in a liquid form by the user after the oral thin film dissolves. The active ingredients are delivered to the gastrointestinal tract of the user where they are absorbed into the systemic circulation. The zinc compound may be microencapsulated to prevent the metallic taste of the zinc compound.

### TECHNICAL FIELD

The present invention relates to oral formulations of zinc, lutein, and zeaxanthin and, more particularly, to formulations of zinc, lutein, and zeaxanthin in rapidly dissolving, ingestible oral thin films for delivery of zinc, lutein, and zeaxanthin to the gastrointestinal tract.

### BACKGROUND ART

Macular degeneration of the retina is associated with aging and the appearance of yellow deposits (drusen) in the retina. Age-related macular degeneration breaks down cells in the retina and is now a major cause of blindness in the United States for individuals over 65 years of age. About 2 million Americans have advanced age-related macular degeneration and another 8 million are at risk. Individuals so afflicted can anticipate either a progressive deterioration of vision or at times a relatively static course, but no spontaneous improvement, since the basic architecture of the retina is destroyed.

Dietary supplements are taken for a variety of reasons including the prophylaxis of vision loss. Dietary supplements are generally in the form of powders, tablets, capsules or gel-caps and comprise a variety of vitamins, minerals, and herbal or other organic constituents. Studies have suggested that the inclusion of xanthophylls and other carotenoids in dietary supplements may provide superior dietary supplements useful in enhancing the health of the eye. Studies have shown the selective uptake of the carotenoids, zeaxanthin and lutein, by the macula of the eye and that zeaxanthin and its isomer lutein may be beneficial in improving the health of the macula. Zinc is also known to be important to the health of the retina and is useful in retarding macular degeneration changes.

Oral thin films for drug delivery are known in the art and these films are commercially available (for example, J-Pac Medical, Somersworth, N.H., US). Oral thin film drug delivery uses a dissolving film (strip) to administer drugs via absorption in the mouth (buccally, lingually, and/or sublingually). The thin film strips are about the size of a postage stamp. The user places the strip on or under the tongue, or along the inside of the cheek. The film strip rapidly dissolves on the tongue or in the buccal cavity when contact with saliva is made. As these strips melt, they release the medications they contain, making them available for absorption in the mouth. This route of administration bypasses “first-pass” metabolism by the liver, enabling the use of smaller doses of substances compared to orally administered tablets and capsules. Thin film drug delivery is a way to administer medicine to children, older adults, and those who find taking tablets and capsules challenging. These oral thin films are useful where gastrointestinal absorption of the ingredients is not feasible. For example, some ingredients may be broken down in the gastrointestinal tract and not delivered to the systemic circulation.

Supplements such as lutein, zeaxanthin, and zinc are generally administered in oral formulations such as tablets and capsules. It is well established that these supplements reduce vision loss in macular degeneration (see NIH Multicenter Trial AREDS II, JAMA, May 15, 2013-Vol 309, No. 19 p. 2005). These supplements are not always preferred for the elderly who often have trouble swallowing these formulations. Fast dissolving orally consumable thin films having pharmaceutically active agents are known (see, for example, U.S. Pat. No. 6,596,298 to Leung et al.). They are useful for delivering various active ingredients by buccal, lingual, or sublingual absorption. Delivery of supplements such as lutein, zeaxanthin, and zinc into the gastrointestinal tract by fast dissolving orally consumable thin films is not known.

### DISCLOSURE OF THE INVENTION

This invention provides a composition which has a zinc compound, lutein, or zeaxanthin or a combination thereof contained in an ingestible oral thin film in effective amounts. The oral thin film is formulated to dissolve in the mouth of a user within 15 seconds to 15 minutes. The zinc compound is in the form of zinc acetate, zinc citrate, zinc gluconate, or zinc oxide. Preferably, the zinc compound is in the amount of 15 to 100 mg, the lutein is in the amount of 1 to 20 mg, and the zeaxanthin is in the amount of 0.5 to 4 mg. Each of these is contained in a liquid based film layer of the oral thin film. The liquid based layer is combined with a powder matrix layer. The zinc compound is, preferably, contained in microcapsules. The microcapsules are formulated to dissolve 5 to 10 minutes after the oral thin film dissolves in the mouth of a user.

The active ingredients of a zinc compound, lutein, and zeaxanthin are useful in inhibiting the progression of macular degeneration and are, preferably, used in combination. The ingestible oral thin film of the present invention is particularly useful in this regard. The ingestible oral thin film having a zinc compound, lutein, or zeaxanthin or a combination thereof contained therein in effective amounts is placed in the mouth of a user. The oral thin film is allowed to dissolve in the mouth of the user. After the oral thin film dissolves the zinc compound, lutein, and/or zeaxanthin are ingested (swallowed) by the user. The zinc compound, lutein, and/or zeaxanthin are then delivered to the gastrointestinal tract of the user where they are available for systemic absorption.

An advantage of the present invention is a formulation of a zinc compound, lutein, and zeaxanthin in an ingestible, dissolvable oral thin film which is easy to swallow, compared to commonly used pills, tablets, and capsules.

Another advantage is a formulation of active ingredients of a zinc compound, lutein, and zeaxanthin in an ingestible, dissolvable oral thin film which provides excellent absorption of these active ingredients from the gastrointestinal tract into the systemic circulation.

Another advantage is the formulation of the zinc compound into microcapsules so that there is no metallic taste from the zinc because the microcapsules do not dissolve until after they are swallowed.

### BEST MODES FOR CARRYING OUT THE INVENTION

This application claims priority to U.S. Provisional Patent Application No. 61/903,655 filed on Nov. 13, 2013, the disclosure of which is incorporated herein by reference. While the following description details the preferred embodiments of the present invention, it is to be understood that the invention is not limited in its application to the details of the oral thin film or method described herein, since the invention is capable of other embodiments and of being practiced in various ways.

The present invention is an oral thin film containing the active ingredients zinc, lutein, or zeaxanthin or combinations thereof to slow the progression of macular degeneration. The oral thin film is placed in the mouth and the ingredients are released from the oral thin film as the oral thin film dissolves in the mouth by the action of saliva. The ingredients in the saliva are swallowed and are then available for systemic absorption in the gastrointestinal tract.

Formulations of oral drug films or strips include natural strip-forming polymers, plasticizers, active pharmaceutical ingredients, sweetening agents, saliva-stimulating agents, flavoring agents, coloring agents, stabilizing and thickening agents. A preferred embodiment is a film or strip having two layers. The first layer is a liquid-based film layer that contains and stabilizes the active ingredients. This may be combined with a second layer which is a powder matrix. This powder matrix provides improved stability as well as additional active ingredients. It may also provide other features such as extra flavoring ingredients, pliability enhancers, and mucosal permeation enhancers. In a two layer strip, the layers are designed to work together. The powder composition can be varied, as can the muco-adhesion properties of the strips, to alter the dissolution and absorption rates of the active ingredient(s).

Any suitable kind of oral thin film can be used in the present invention. U.S. Patent Application Publication Nos. US 2006/0210610 and US 2011/0305768, the disclosures of which are incorporated herein by reference, disclose detailed methods for making oral thin films and incorporating active ingredients and excipients therein. The film may be a clear or opaque, flexible, thin material. Typical thicknesses range from 0.01 to 2 mm. The film may have any suitable shape, including round, oval, rectangle, or square. The film may be a monolayer, bilayer or trilayer film. In the preferred embodiment, the film is designed to be suitable for buccal mucosal administration. The monolayer film contains an active agent and one or more excipients. The bilayer film contains an active agent in the first layer and one or more excipients, such as a solubilizing agent, in the second layer. This configuration allows the active agent to be stored separated from the excipients, may increase the stability of the active agent, and may increase the shelf life of the composition compared to if the excipients and active agent were contained in a single layer. The trilayer film contains three layers of film. Each of the layers may be different, or two of the layers, such as the bottom and top layers, may have substantially the same composition. In one embodiment, the bottom and top layers surround a core layer containing the active agent(s). The bottom and top layers may contain one or more excipients. Preferably, the bottom and top layers have the same composition. Alternatively, the bottom and top layers may contain different excipient(s), or different amounts of the same excipient(s). The core layer typically contains the active agent(s), optionally with one or more excipients. By altering the composition of the excipients, the film can be designed to dissolve rapidly (less than 30 seconds) or slowly (up to 15 minutes) in order to achieve the desired absorption profile and subsequent effect. The film may dissolve in a time period ranging from 3 to 5 minutes, 5 to 8 minutes, or 8 to 12 minutes. Preferably, the film dissolves in a time period ranging from 15 seconds to 2 minutes.

In the preferred embodiment of oral thin film formulations the active ingredients of zinc, lutein, or zeaxanthin or combinations thereof are contained in a liquid based film layer. The preferred combinations are zinc alone, lutein plus zeaxanthin, and lutein plus zeaxanthin plus zinc. Zinc is in the amount of 15 to 100 mg of, preferably, the form of zinc acetate, oxide, citrate or gluconate. Lutein (lutein esters) is in the amount of 1 to 20 mg, preferably 10 mg. Zeaxanthin (zeaxanthin esters) is in the amount of 0.5 to 4 mg, preferably 2 mg. The liquid based film layer is combined with a powder matrix layer by methods known in the art. Active ingredients may also include Vitamin C, 50 to 500 mg, preferably 125 mg, and Vitamin E, 30 IU to 400 IU, preferably 100 IU. Other ingredients can include pectin, water, glycerin, sucralose, polysorbate 60, microcrystalline cellulose, monoammonium glycyrrhizinate, lecithin, cocoa butter, natural crystal white flavor oil, talc, chitosan, genuvisco carrageenan, menthol, and flavors. The powder matrix layer provides, for example, pliability enhancers, mucosal permeation enhancers, and stabilizers which are all well known in the art (see, for example, Patent Application Publication No. US 2006/0210610). If desired, the powder matrix layer can contain flavoring agents and other active ingredients.

Zinc salts or zinc oxide can have a metallic taste which may be objectionable and difficult to mask. The formulation of the present invention includes zinc salts or oxide enclosed within a microcapsule. Zinc can be microencapsulated by methods well known in the art. See, for example, U.S. Patent Application Publication Nos. US 2006/0210610 and US 2011/0305768. The microcapsules are constructed to dissolve within minutes, but after a period of time sufficient for a user to swallow the ingredients of the dissolved oral thin film. For example, the oral thin film will dissolve in the mouth within 15 to 60 seconds but the capsules are formulated to dissolve 5 to 10 minutes after the oral thin film dissolves. The metallic taste of zinc is not detected when the zinc is microencapsulated. Once the microcapsules are swallowed before they dissolve, there is no possibility of a metallic zinc taste. The active ingredients in the oral thin film enter the gastrointestinal tract where they are absorbed into the systemic circulation.

The present invention provides a method for slowing the progression of macular degeneration. The method comprises the following steps. 1) providing an oral thin film having the active ingredients contained therein as described above; 2) placing the oral thin film in the mouth of a user; 3) allowing the oral thin strip to dissolve in the mouth of the user; and 4) ingesting the active ingredients by the user, thereby delivering the active ingredients to the gastrointestinal tract of the user. If the zinc ingredient is microencapsulated, then the method includes the step of ingesting (swallowing) the active ingredients before the microcapsules containing the zinc ingredient dissolve.

### EXAMPLE

The bioavailability after ingestion of zinc in an oral thin film strip (also containing lutein and zeaxanthin), was compared to the bioavailability after ingestion of zinc in a capsule (also containing lutein and zeaxanthin). The oral thin film and the capsule were prepared by Cure Pharmaceuticals Corporation, Oxnard, Calif., US. Both the oral thin film and the capsule contained zinc 33.3 mg, lutein 9 mg, and zeaxanthin 4.8 mg.

Twelve New Zealand White Rabbits were randomly allotted to 2 groups (6 animals/group). The animals were fasted overnight and water was provided ad libitum

Each animal was anaesthetized by using 4% v/v of isoflurane anesthesia for 5 minutes for dose administration followed by 2% v/v isoflurane as maintenance for the following 5 min. The test item strip was placed on the tongues of the anesthetized Group 1 rabbits followed by 3 mL of distilled water to facilitate swallowing. The capsule was administered by placing a capsule on the base of the tongues of the Group 2 rabbits followed by 3 mL of distilled water to facilitate swallowing.

Approximately 1.0 mL of blood was collected from the marginal ear vein of each rabbit into pre-labeled tubes containing K2-EDTA from each rabbit at 0 (pre-dose), 0.25, 0.50, 1.0, 2.0, 3.0, 3.5 and 4.0 hours post dose. Blood was centrifuged at 5000 rpm for 10 min at 4° C. and the plasma was separated into labeled micro-tubes and made into two aliquots. One aliquot was transported in dry ice storage condition to Auriga Research,

Bangalore, for Zinc analysis. Pharmacokinetic parameters of plasma Zinc content were determined by non-compartmental analysis model using PKsolver. The results are shown in the table below:

Pharmacokinetic Parameters of the Strip and Capsule

| | | Group 1 | Group 2 |
|---|---|---|---|
| PK Parameters | | Oral thin film | Capsule |
| Cₘₐₓ | Mean | 2.83 | 2.77 |
| (ppm) | ±SD | 0.54 | 0.65 |
| | n | 6 | 6 |
| Tₘₐₓ | Median | 3.25 | 2.50 |
| (hr) | Minimum | 2.00 | 0.25 |
| | Maximum | 4.00 | 3.50 |
| AUC₀₋ₜ | Mean | 7.96 | 7.93 |
| (ppm × h) | ±SD | 1.72 | 1.59 |
| | n | 6 | 6 |

Cmax = The maximum concentration of zinc in the plasma
Tmax = Time to reach the maximum concentration
AUC0-4 h = Area under the concentration-time curve from zero to 4 hr
The endogenous zinc levels (zero hour) were similar between the two treatment groups (mean zero hour values were 1.83 and 1.98 ppm, respectively for oral thin film and the capsule formulations.

The bioavailability of zinc in the oral thin film was not significantly different from that in the capsule. In rabbits receiving the oral thin film, 19 out of 42 plasma samples (45%) had plasma levels of zinc higher than the endogenous level of zinc. In contrast, in rabbits receiving the capsule, only 13 out of 42 plasma samples (31%) had plasma levels of zinc higher than the endogenous level of zinc. Furthermore, with the oral thin films, plasma levels increased to a maximum above endogenous levels 1.00±0.15 ppm (mean±SE). In contrast, the increase was only 0.78+0.13 ppm with the capsules. The oral thin film produced a 28% greater maximum increase in plasma levels of zinc compared to the capsule.

The data show that the ingredients of the oral thin film formulation of zinc, lutein, and zeaxanthin reached the gastrointestinal tract for systemic absorption just as well as, if not better than, the capsule formulation. Thus, the oral thin film preparation of the present invention provides an improved method of oral dosing with zinc, lutein, and zeaxanthin which is more acceptable to people who have difficulty in swallowing pills and capsules. The microencapsulated form of zinc provides an effective method of making zinc palatable without the use of masking agents while still allowing good systemic absorption of zinc.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. For example, the ingestible oral thin film can be any type known in the art as long as it is compatible with zinc compounds, microencapsulated zinc compounds, lutein, and zeaxanthin. Any type of encapsulation formulation known in the art can be used with zinc compounds as long as the capsule does not dissolve before a user has time to swallow the ingredients of the dissolved ingestible oral thin film. Any type of flavoring may be used in the oral thin film, preferably mint flavor.

It will be understood that various changes in the details, materials, and arrangements of the parts which have been described above in order to explain the nature of this invention may be made by those skilled in the art without departing from the principle and scope of the invention as described and claimed herein.

1. An oral thin film for inhibiting the progression of macular degeneration, comprising a zinc compound, lutein, or zeaxanthin, or a combination thereof, contained in an ingestible oral thin film in effective amounts.

2. The oral thin film of claim 1 wherein said oral thin film is formulated to dissolve in the mouth of a user within 15 seconds to 15 minutes.

3. The oral thin film of claim 1 wherein said zinc compound is in the form of zinc acetate, zinc citrate, zinc gluconate, or zinc oxide.

4. The oral thin film of claim 1 wherein said zinc compound is in the amount of 15 to 100 mg, said lutein is in the amount of 1 to 20 mg, and said zeaxanthin is in the amount of 0.5 to 4 mg, each contained in a liquid based film layer of said oral thin film, said liquid based layer being combined with a powder matrix layer.

5. The oral thin film of claim 1 wherein said zinc compound is contained in microcapsules.

6. The oral thin film of claim 5 wherein said microcapsules are formulated to dissolve 5 to 10 minutes after said oral thin film dissolves in the mouth of a user.

7. An oral thin film for inhibiting the progression of macular degeneration, comprising a zinc compound, lutein, and zeaxanthin contained in an ingestible oral thin film, wherein said oral thin film is formulated to dissolve in the mouth of a user within 15 seconds to 15 minutes and wherein said zinc compound is in the form of zinc acetate, zinc citrate, zinc gluconate, or zinc oxide.

8. The oral thin film of claim 7 wherein said zinc compound is in the amount of 15 to 100 mg, said lutein is in the amount of 1 to 20 mg, and said zeaxanthin is in the amount of 0.5 to 4 mg, each contained in a liquid based film layer of said oral thin film, said liquid based layer being combined with a powder matrix layer.

9. The oral thin film of claim 8 wherein said zinc compound is contained in microcapsules.

10. The oral thin film of claim 9 wherein said microcapsules are formulated to dissolve 5 to 10 minutes after said oral thin film dissolves in the mouth of a user.

11. A method of inhibiting the progression of macular degeneration, comprising the steps of:
1) providing an ingestible oral thin film comprising a zinc compound, lutein, or zeaxanthin, or a combination thereof, contained in an ingestible oral thin film in effective amounts;
2) placing said oral thin film in the mouth of a user;
3) allowing said oral thin film to dissolve in the mouth of the user;
4) ingesting said zinc compound, said lutein, or said zeaxanthin, or said combination thereof, by the user after said oral thin film dissolves; and
5) delivering said zinc compound, said lutein, or said zeaxanthin, or said combination thereof, to the gastrointestinal tract of the user after ingesting said zinc compound, said lutein, or said zeaxanthin, or said combination thereof.

12. The method of claim 11 wherein said oral thin film is formulated to dissolve in the mouth of the user within 15 seconds to 15 minutes.

13. The method of claim 11 wherein said zinc compound is in the form of zinc acetate, zinc citrate, zinc gluconate, or zinc oxide.

14. The method of claim 11 wherein said zinc compound is in the amount of 15 to 100 mg, said lutein is in the amount of 1 to 20 mg, and said zeaxanthin is in the amount of 0.5 to 4 mg, each contained in a liquid based film layer of said oral thin film, said liquid based layer being combined with a powder matrix layer.

15. The method of claim 11 wherein said zinc compound is contained in microcapsules.

16. The method of claim 15 wherein said microcapsules are formulated to dissolve 5 to 10 minutes after said oral thin film dissolves in the mouth of the user.

17. A method of inhibiting the progression of macular degeneration, comprising the steps of:
1) providing an ingestible oral thin film comprising a zinc compound, lutein, and zeaxanthin contained in an ingestible oral thin film in effective amounts;
2) placing said oral thin film in the mouth of a user;
3) allowing said oral thin film to dissolve in the mouth of the user;
4) ingesting said zinc compound, said lutein, and said zeaxanthin by the user after said oral thin film dissolves; and
5) delivering said zinc compound, said lutein, and said zeaxanthin to the gastrointestinal tract of the user after ingesting said zinc compound, said lutein, and said zeaxanthin, wherein said oral thin film is formulated to dissolve in the mouth of the user within 15 seconds to 15 minutes and wherein said zinc compound is in the form of zinc acetate, zinc citrate, zinc gluconate, or zinc oxide.

18. The method of claim 17 wherein said zinc compound is in the amount of 15 to 100 mg, said lutein is in the amount of 1 to 20 mg, and said zeaxanthin is in the amount of 0.5 to 4 mg, each contained in a liquid based film layer of said oral thin film, said liquid based layer being combined with a powder matrix layer.

19. The method of claim 18 wherein said zinc compound is contained in microcapsules.

20. The method of claim 19 wherein said microcapsules are formulated to dissolve 5 to 10 minutes after said oral thin film dissolves in the mouth of the user.
